(19) United States
(12) Patent Application Publication
(10) Pub. No.: US 2013/0281854 A1
(43) Pub. Date: Oct. 24, 2013

(54) DIAGNOSTIC SYSTEM AND METHOD FOR OBTAINING DATA RELATING TO A CARDIAC MEDICAL CONDITION

(71) Applicants: Susan Martignetti Stuebe, Whitefish Bay, WI (US); Daniel Lee Eisenhut, Hartland, WI (US); William Kohn, Madison, WI (US); Kelley Webster, Hartford, WI (US); Srinivas Paladugu, Knoxville, TN (US); Roxanne A. Ludwigson, Milwaukee, WI (US)

(72) Inventors: Susan Martignetti Stuebe, Whitefish Bay, WI (US); Daniel Lee Eisenhut, Hartland, WI (US); William Kohn, Madison, WI (US); Kelley Webster, Hartford, WI (US); Srinivas Paladugu, Knoxville, TN (US); Roxanne A. Ludwigson, Milwaukee, WI (US)

(73) Assignee: GENERAL ELECTRIC COMPANY, Schenetady, NY (US)

(21) Appl. No.: 13/454,945

(22) Filed: Apr. 24, 2012

(51) Int. Cl. A61B 8/13; A61B 5/044

(52) U.S. Cl. 600/440; 600/443

(57) ABSTRACT

A medical diagnostic system is provided that includes an electrocardiograph (ECG) device and an ultrasound imaging device. The diagnostic system also includes a user interface having a display. The user interface is configured to receive operator inputs from an operator of the diagnostic system, wherein the user interface is configured to show on the display a plurality of different screens to the operator during a workflow. The screens include user-selectable elements that are configured to be activated by the operator during the workflow. The user interface is configured to display the different screens to the operator in a predetermined manner to guide the operator through the workflow to obtain an electrocardiogram of the heart of the patient and an ultrasound image of the heart. The user interface also configured to guide the operator to obtain structural measurements of the heart based on the ultrasound image.

### BACKGROUND OF THE INVENTION

The subject matter herein relates generally to systems and methods for obtaining data relating to a patient's health and/or anatomy, and more particularly, to systems and methods that are configured to obtain data relating to cardiac function and/or cardiac structures.

An electrocardiogram (ECG) is a recording of the combined electrical activity of the cells of the heart (or cardiac cells). During a heartbeat, the cardiac cells experience electrical impulses called action potentials that cause the cardiac cells to contract. The combined electrical activity of the cardiac cells detected by the electrodes during the cardiac cycle may be processed into a waveform that shows electrical potential over time. One conventional waveform for a complete heartbeat includes a P wave, a QRS complex, and a T wave. The P wave is associated with atrial contraction, the QRS complex describes ventricular contraction, and the T wave describes ventricular de-contraction.

The recorded waveform, which may be referred to as the ECG, can inform a doctor or other healthcare provider about the heart of the patient. For example, ECGs may be used to diagnose a medical condition of the heart, such as arrhythmia, ischemia, infarction, cardiomyopathy, or other electrophysiological abnormalities. As a specific example, ECGs may be used to diagnose left-ventricular hypertrophy (LVH), which is indicative of hypertrophic cardiomyopathy (HCM).

Another diagnostic tool used by healthcare providers includes ultrasound images. Ultrasound imaging can provide images of subcutaneous structures, including the heart. Ultrasound images of the heart (also called echocardiograms or “echos”) may show anatomical structures (e.g., ventricles, atria, valves, septum, and the like) as well as blood flow through the heart. An ultrasound image of the heart may be used to measure dimensions of designated structures of the heart to diagnose a medical condition. For example, cardiovascular mortality and morbidity increases with increasing values of left ventricular (LV) mass. LVH is a thickening of the myocardium of the left ventricle. Accordingly, ultrasound images of the left ventricle may be analyzed to determine whether the left ventricle has an increased LV mass and/or LVH.

However, the process of obtaining an ECG and the process of obtaining an echocardiogram are typically performed by different technicians who have received specialized training for the particular diagnostic tool. Conventional methods of obtaining ECGs use multiple electrodes (e.g., three, ten) that are placed on the skin of a patient in designated locations. Conventional echocardiography includes the careful application and manipulation of an ultrasound probe and a computer interface to obtain the desired ultrasound image. Thus, different systems are used for obtaining ECGs and ultrasound images, which can add time and complexity to the acquisition and review process.

### BRIEF DESCRIPTION OF THE INVENTION

In one embodiment, a medical diagnostic system is provided that includes an electrocardiograph (ECG) device having at least one electrode that is configured to obtain electrical data for a heart of a patient. The diagnostic system also includes an ultrasound imaging device having an ultrasound probe that is configured to obtain ultrasound data of the heart of the patient. The diagnostic system also includes a user interface having a display. The user interface is configured to receive operator inputs from an operator of the diagnostic system, wherein the user interface is configured to show on the display a plurality of different screens to the operator during a workflow. The screens include user-selectable elements that are configured to be activated by the operator during the workflow. The user interface is configured to display the different screens to guide the operator through the workflow to obtain the electrical data and the ultrasound data. The user interface also configured to guide the operator to obtain structural measurements of the heart based on the ultrasound data.

In another embodiment, a medical diagnostic system is provided that includes an ultrasound imaging device having an ultrasound probe that is configured to obtain ultrasound data of a heart of a patient. The diagnostic system also includes a cardiac cycle analyzer that is configured to analyze the ultrasound data to automatically identify a cardiac-cycle image from a set of ultrasound images based on the ultrasound data. The cardiac-cycle image includes the heart at a predetermined cardiac-cycle event. The diagnostic system also includes a measurement module that is configured to analyze the cardiac-cycle image and automatically position a reference object relative to at least one anatomical structure of the heart in the cardiac-cycle image. The diagnostic system also includes a user interface having a display configured to display the reference object and the cardiac-cycle image. The user interface is configured to receive operator inputs to at least one of (a) designate, from the set of ultrasound images, a different ultrasound image as the cardiac-cycle image or (b) re-position the reference object relative to the at least one anatomical structure.

In another embodiment, a method of obtaining measurements of a heart of a patient is provided. The method includes automatically identifying a cardiac-cycle image from a set of ultrasound images. The cardiac-cycle image includes the heart at a predetermined cardiac-cycle event. The method also includes displaying the cardiac-cycle image to an operator using a user interface display. The method also includes automatically positioning a reference object relative to at least one anatomical structure of the heart in the cardiac-cycle image. The reference object is positioned to obtain designated measurements of the heart. The method also includes receiving operator inputs from the operator to at least one of (a) designate, from the set of ultrasound images, a different ultrasound image as the cardiac-cycle image or (b) re-position the reference object relative to the at least one anatomical structure. The method also includes determining at least one measurement of the heart using the reference object and the cardiac-cycle image.

### BRIEF DESCRIPTION OF THE DRAWINGS

FIG. 1 is a block diagram of a diagnostic system formed in accordance with one embodiment for obtaining at least one of an electrocardiogram (ECG) or an ultrasound image.

FIG. 2 is a flow chart that illustrates a workflow in accordance with one embodiment that may be performed with the diagnostic system of FIG. 1.

FIG. 3 shows a demographic screen that may be displayed by the diagnostic system of FIG. 1.

FIG. 4 shows another demographic screen that may be displayed by the diagnostic system of FIG. 1.

FIG. 5 shows an ECG-acquisition screen that may be displayed by the diagnostic system of FIG. 1.

FIG. 6 illustrates graphical-user-interface elements that may be utilized by the diagnostic system of FIG. 1 to assist the operator for a diagnostic session.

FIG. 7 shows an ultrasound-acquisition screen that may be displayed by the diagnostic system of FIG. 1.

FIG. 8 shows another ultrasound-acquisition screen that may be displayed by the diagnostic system of FIG. 1.

FIG. 9 shows a measurement screen that may be displayed by the diagnostic system of FIG. 1.

FIG. 10 shows another measurement screen that may be displayed by the diagnostic system of FIG. 1.

FIG. 11 shows another measurement screen that may be displayed by the diagnostic system of FIG. 1.

FIG. 12 is a perspective view of a portable diagnostic system formed in accordance with one embodiment.

### DETAILED DESCRIPTION OF THE INVENTION

Exemplary embodiments that are described in detail below provide systems and methods for obtaining at least one of an electrocardiogram (ECG) or a medical image, such as an ultrasound image. In some embodiments, both an ECG and an ultrasound image are obtained and, more particularly, an ECG and ultrasound image of a patient heart are obtained. Embodiments described herein may include systems and methods for obtaining data relating to a heart of a patient that may be used to diagnose a medical condition of the heart. For example, one or more embodiments may be used to determine dimensions of anatomical structures in the heart. An exemplary medical condition that may be diagnosed by one or more embodiments is left-ventricular hypertrophy (LVH). Embodiments may also be used to provide information to a qualified doctor or other individual that may assist the doctor in diagnosing hypertension in a patient. However, embodiments described herein may assist in diagnosing other medical conditions.

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

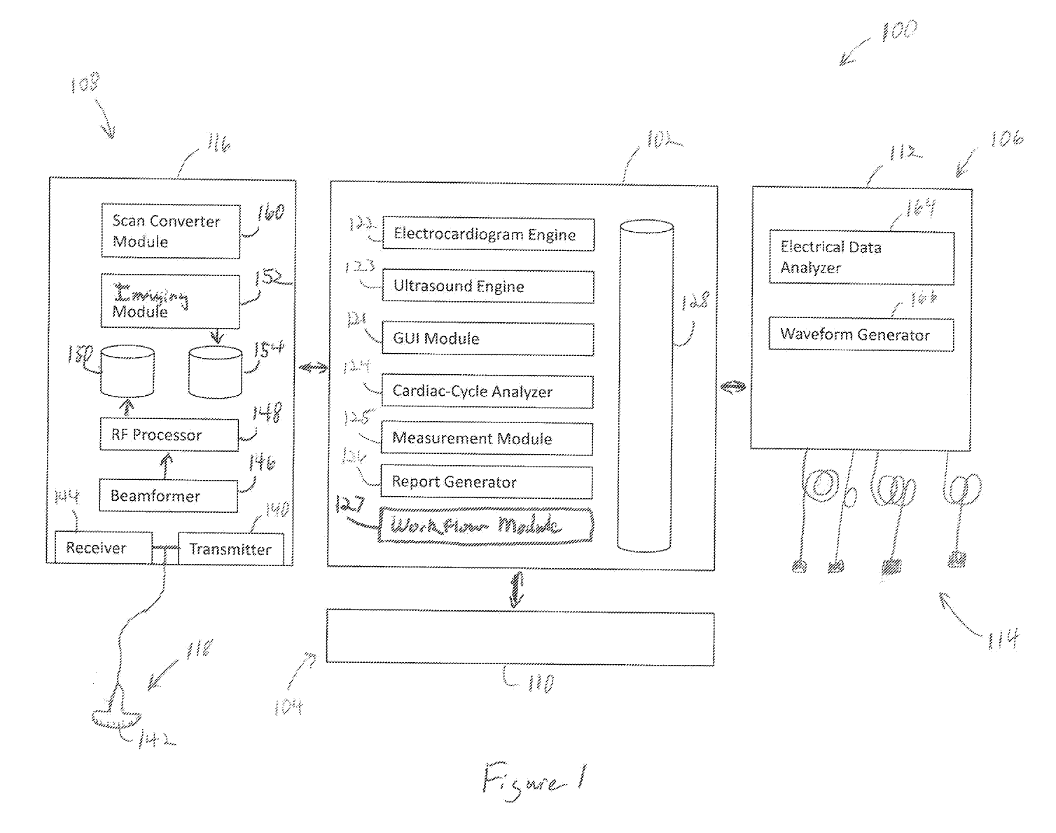

FIG. 1 is a block diagram of a medical diagnostic system 100 formed in accordance with one embodiment for obtaining at least one of an electrocardiogram (ECG) or an ultrasound image. The diagnostic system 100 includes a computing system 102, a user interface 104, an electrocardiogram (ECG) monitor or device 106, and an ultrasound imaging device 108. The computing system 102 is communicatively coupled to the user interface 104 and the ECG and imaging devices 106, 108 and is configured to control operation of the user interface 104 and the ECG and imaging devices 106, 108. In one embodiment, the ECG and imaging devices 106, 108 form sub-systems of the diagnostic system 100.

In an exemplary embodiment, the computing system 102 includes one or more processors/modules configured to instruct the user interface 104 and the ECG and imaging devices 106, 108 to operate in a designated manner during, for example, a diagnostic session. The computing system 102 is configured to execute a set of instructions that are stored in one or more storage elements (e.g., instructions stored on a tangible and/or non-transitory computer readable storage medium) to control operation of the diagnostic system 100. The set of instructions may include various commands that instruct the computing system 102 as a processing machine to perform specific operations such as the workflows, processes, and methods described herein. In FIG. 1, the computing system 102 is indicated as a separate unit with respect to the user interface 104 and the ECG and imaging devices 106, 108. However, it is understood that computing system 102 is not necessarily separate from the user interface 104 and the ECG and imaging devices 106, 108. Instead, the computing system 102 may be distributed in parts of the user interface 104 and/or the ECG and imaging devices 106, 108.

The user interface 104 may include hardware, firmware, software, or a combination thereof that enables an individual (e.g., an operator) to directly or indirectly control operation of the diagnostic system 100 and the various components thereof. As shown, the user interface 104 includes a user display 110. In some embodiments, the user interface 104 may also include one or more input devices (not shown), such as a physical keyboard, mouse, and/or touchpad. In an exemplary embodiment, the display 110 is a touch-sensitive display (e.g., touchscreen) that can detect a presence of a touch from an operator of the diagnostic system 100 and can also identify a location in the display area of the touch. The touch may be applied by, for example, at least one of an individual's hand, glove, stylus, or the like. As such, the touch-sensitive display may receive inputs from the operator and also communicate information to the operator.

The ECG device 106 includes a base unit 112 and a plurality of electrodes 114 (or leads) that are communicatively coupled to the base unit 112. The imaging device 108 includes a base unit 116 and an ultrasound probe or transducer 118. The computing system 102, the user interface 104, the ECG and imaging devices 106, 108 may be constructed into a single device or apparatus. For example, the computing system 102, the user interface 104, and the base units 112, 116 may be integrated into one component that is communicatively coupled to the probe 118 and the electrodes 114. For example, the integrated component may be similar to a tablet computer, a laptop computer, or desktop computer. Alternatively, the diagnostic system 100 may be several components that may or may not be located near each other. In some embodiments, the base units 112, 116 share a common housing as shown in the portable diagnostic system 600 shown in FIG. 12.

As used herein, an anatomical structure may be an entire organ or system or may be an identifiable region or structure within the organ or system. In particular embodiments, the anatomical structures that are analyzed are structures of the heart. Examples of anatomical structures of the heart include, but are not limited to, the epicardium, endocardium, mid-myocardium, one or both atria, one or both ventricles, walls of the atria or ventricles, valves, a group of cardiac cells within a predetermined region of the heart, and the like. In particular embodiments, the anatomical structures include the septal and posterior walls of the left ventricle. However, in other embodiments, anatomical structures may be structures found elsewhere in the body of the patient, such as other muscles or muscle systems, the nervous system or identifiable nerves within the nervous system, organs, and the like. It should also be noted that although the various embodiments may be described in connection with obtaining data related to a patient that is human, the patient may also be an animal.

As used herein, “communicatively coupled” includes devices or components being electrically coupled to each other through, for example, wires or cables and also includes devices or components being wirelessly connected to each other such that one or more of the devices or components of the diagnostic system 100 may be located remote from the others. For example, the user interface 104 may be located at one location (e.g., hospital room or research laboratory) and the computing system 102 may be remotely located (e.g., central server system).

As used herein, a “diagnostic session” is a period of time in which an operator uses the diagnostic system 100 to prepare for and/or obtain data from a patient that may be used to diagnose a medical condition. During a diagnostic session, the operator may use at least one of the user interface 104 to enter patient information, the ECG device 106, the imaging device 108, or other biomedical device. By way of example, a diagnostic session may include coupling the electrodes 114 to a patient's body, applying gel to the patient's body for ultrasound imaging, capturing ultrasound images using the probe 118, and interacting with the user interface 104 to obtain the diagnostic data of the patient. The diagnostic data may include at least one of an ECG recording (or reading), an ultrasound image, or a measurement derived from the ECG recording and/or ultrasound image. The ultrasound image may include a view of the heart when the heart is in a designated orientation with respect to the ultrasound probe. When the heart is in the designated orientation, one or more structural measurements of the heart may be determined from the corresponding ultrasound image. The structural measurements determined may include dimensions (e.g., thickness), volume, area, and the like. Other measurements may be computed from the structural measurements that are obtained from the ultrasound image(s).

As used herein, a “predetermined cardiac-cycle event” may be an identifiable stage or moment in the cardiac cycle. In some cases, the stage or moment may occur when various structures of the heart have a relative position with respect to each other. For example, the stage or moment may occur when two walls have a greatest separation distance therebetween or a least separation distance therebetween (e.g., when a portion of the heart is contracted). As another example, the stage or moment may occur when a valve is fully opened or closed. The predetermined cardiac-cycle event may also be determined by analyzing the electrical activity of the heart (e.g., the ECG). In particular embodiments, the predetermined cardiac-cycle event is an end diastole of the cardiac cycle.

As used herein, a “user-selectable element” includes an identifiable element that is configured to be activated by an operator. The user-selectable element may be a physical element of an input device, such as a keyboard or keypad, or the user-selectable element may be a graphical-user-interface (GUI) element (e.g., a virtual element) that is displayed on a screen. User-selectable elements are configured to be activated by an operator during a diagnostic session. Activation of the user-selectable element may be accomplished in various manners. For example, the user-selectable element (physical or virtual) may be pressed by the operator, selected using a cursor and/or a mouse, selected using keys of a keyboard, voice-activated, and the like. By way of example, the user-selectable element may be a key of a keyboard (physical or virtual), a tab, a switch, a lever, a drop-down menu that provides a list of selections, a graphical icon, and the like. In some embodiments, the user-selectable element is labeled or otherwise differentiated (e.g., by drawing or unique shape) with respect to other user-selectable elements. When a user-selectable element is activated by an operator, signals are communicated to the diagnostic system 100 (e.g., the computing system 102) that indicate the operator has selected and activated the user-selectable element and, as such, desires a predetermined action. The signals may instruct the diagnostic system 100 to act or respond in a predetermined manner.

In some embodiments, the diagnostic system 100 may be activated by user motions without specifically engaging a user-selectable element. For example, the operator of the diagnostic system 100 may engage the screen by quickly tapping, pressing for longer periods of time, swiping with one or more fingers (or stylus unit), or pinching the screen with multiple fingers (or styluses). Other gestures may be recognized by the screen. In other embodiments, the gestures may be identified by the diagnostic system 100 without engaging the screen. For example, the diagnostic system 100 may include a camera (not shown) that monitors the operator. The diagnostic system 100 may be programmed to respond when the operator performs predetermined motions.

The imaging device 108 includes a transmitter 140 that drives an array of transducer elements 142 (e.g., piezoelectric crystals) within the probe 118 to emit pulsed ultrasonic signals into a body or volume. The pulsed ultrasonic signals may be for imaging of a ROI that includes an anatomical structure, such as a heart. The ultrasonic signals are back-scattered from structures in the body, for example, adipose tissue, muscular tissue, blood cells, veins or objects within the body (e.g., a catheter or needle) to produce echoes that return to the transducer elements 142. The echoes are received by a receiver 144. The received echoes are provided to a beamformer 146 that performs beamforming and outputs an RF signal. The RF signal is then provided to an RF processor 148 that processes the RF signal. Alternatively, the RF processor 148 may include a complex demodulator (not shown) that demodulates the RF signal to form IQ data pairs representative of the echo signals. The RF or IQ signal data may then be provided directly to a memory 150 for storage (e.g., temporary storage).

The imaging device 108 may also include a processor or imaging module 152 to process the acquired ultrasound information (e.g., RF signal data or IQ data pairs) and prepare frames of ultrasound information for display. The imaging module 152 is adapted to perform one or more processing operations according to a plurality of selectable ultrasound modalities on the acquired ultrasound information. Acquired ultrasound information may be processed in real-time during a diagnostic session as the echo signals are received. Additionally or alternatively, the ultrasound information may be stored temporarily in the memory 150 during a diagnostic session and processed in less than real-time in a live or off-line operation. An image memory 154 is included for storing processed frames of acquired ultrasound information that are not scheduled to be displayed immediately. The image memory 154 may comprise any known data storage medium, for example, a permanent storage medium, removable storage medium, etc.

The imaging module 152 is communicatively coupled to the user interface 104 that is configured to receive inputs from the operator to control operation of the imaging device 108. The display 110 may automatically display, for example, a 2D, 3D, or 4D ultrasound data set stored in the memory 150 or 154 or currently being acquired. The data set may also be displayed with a graphical representation (e.g., a reference object). One or both of the memory 150 and the memory 154 may store 3D data sets of the ultrasound data, where such 3D data sets are accessed to present 2D and 3D images. For example, a 3D ultrasound data set may be mapped into the corresponding memory 150 or 154, as well as one or more reference planes. The processing of the data, including the data sets, may be based in part on operator inputs, for example, user selections received at the user interface 104.

In some embodiments, the ultrasound data may constitute IQ data pairs that represent the real and imaginary components associated with each data sample. The IQ data pairs may be provided to one or more image-processing modules (not shown) of the imaging module 152, for example, a color-flow module, an acoustic radiation force imaging (ARFI) module, a B-mode module, a spectral Doppler module, an acoustic streaming module, a tissue Doppler module, a C-scan module, and an elastography module. Other modules may be included, such as an M-mode module, power Doppler module, harmonic tissue strain imaging, among others. However, embodiments described herein are not limited to processing IQ data pairs. For example, processing may be done with RF data and/or using other methods.

Each of the image-processing modules may be configured to process the IQ data pairs in a corresponding manner to generate color-flow data, ARFI data, B-mode data, spectral Doppler data, acoustic streaming data, tissue Doppler data, C-scan data, elastography data, among others, all of which may be stored in a memory temporarily before subsequent processing. The image data may be stored, for example, as sets of vector data values, where each set defines an individual ultrasound image frame. The vector data values are generally organized based on the polar coordinate system. A scan converter module 160 may access and obtain from the memory the image data associated with an image frame and convert the image data to Cartesian coordinates to generate an ultrasound image formatted for display.

The ECG device 106 may include an electrical data analyzer 164 and a waveform generator 166. The data analyzer 164 may be configured to analyze the electrical signals detected by the electrodes 114 and verify that the electrical signals from each electrode 114 are accurate for the location of the corresponding electrode 114. More specifically, the data analyzer 164 may facilitate determining if the electrodes are (a) not sufficiently coupled to the patient; (b) improperly located on the patient; and/or (c) are faulty. The waveform generator 166 is configured to receive the electrical signals from the electrodes 114 and process the collective signals into waveform data. The waveform data may be received by the user interface 102 and displayed to the operator as, for example, a PQRST waveform. The waveform data and/or the presentation of the waveform may be based, at least in part, on operator selections.

The computing system 102 includes a plurality of modules or sub-modules that control operation of the diagnostic system 100. For example, the computing system 102 may include the modules 121-127 and a storage system 128 that communicates with at least some of the modules 121-127 and the ECG and imaging devices 106, 108. The graphical user interface (GUI) module 121 may coordinate with the other modules and the ECG and imaging devices 106, 108 for displaying various objects in the display 110. For example, various images of the user-selectable elements, described in greater detail below, may be stored in the storage system 128 and provided to the display 110 by the GUI module 121.

The computing system 102 also includes a workflow module 127. The workflow module 127 may be configured to respond to operator inputs during a workflow of the diagnostic system 100 and instruct the user interface 104 to show different screens to the operator on the display 110. The screens may be shown in a predetermined manner to guide the operator during the workflow. More specifically, the workflow module 127 may command the user interface to show at least some of the screens in a designated order. As one example, during a stage of the workflow (described in greater detail below), the user interface 104 may show different screens to guide the operator to locate a reference object with respect to an ultrasound image of the heart. When the operator activates, for example, “NEXT” or “SAVE” user-selectable elements on a first screen, the workflow module 127 may instruct the user interface to show a predetermined second screen that is configured to follow the first screen in the workflow.

The computing system 102 may include an ECG engine 122 configured to communicate with and control operation of the ECG device 106. The computing system 102 may also include an ultrasound engine 123 that may be configured to control operation of the imaging device 108. The ECG and ultrasound engines 122, 123 may receive operator inputs and communicate the operator inputs to the probe 118 and the ECG device 106.

The computing system 102 may also include a cardiac-cycle analyzer 124 that is configured to analyze ultrasound data. The ultrasound data may be obtained by the imaging device 108 or the ultrasound data may be provided by another source (e.g., database). The cardiac-cycle analyzer 124 may analyze ultrasound images and automatically identify a designated ultrasound image (also called cardiac-cycle image) from a set of ultrasound images based on the ultrasound data. The cardiac-cycle image may include the heart at a predetermined cardiac-cycle event.

A measurement module 125 of the computing system 102 may be configured to analyze the cardiac-cycle image and automatically position a reference object relative to the heart in the cardiac-cycle image. The reference object may assist in acquiring measurements of the heart. In the illustrated embodiment, the reference object is a projection line. However, in other embodiments, the reference object may be any shape that facilitates acquiring measurements from the ultrasound images.

The computing system 102 may also include a report generator 126. The report generator 126 may analyze measurements obtained by the ECG and imaging devices 106, 108 and provide a report that may or may not include a recommended diagnosis. As such, the report generator 126 may also be referred to as a diagnostic module. The measurements analyzed by the report generator 126 may include an ECG recording, the ultrasound images, measurements of the heart in at least one of the ultrasound images, and other patient information. In some embodiments, the report generator 126 does not process or analyze the measurements, but simply generates a report that includes the measurements in a predetermined format. In some embodiments, the report is a virtual report stored in the diagnostic system 100.

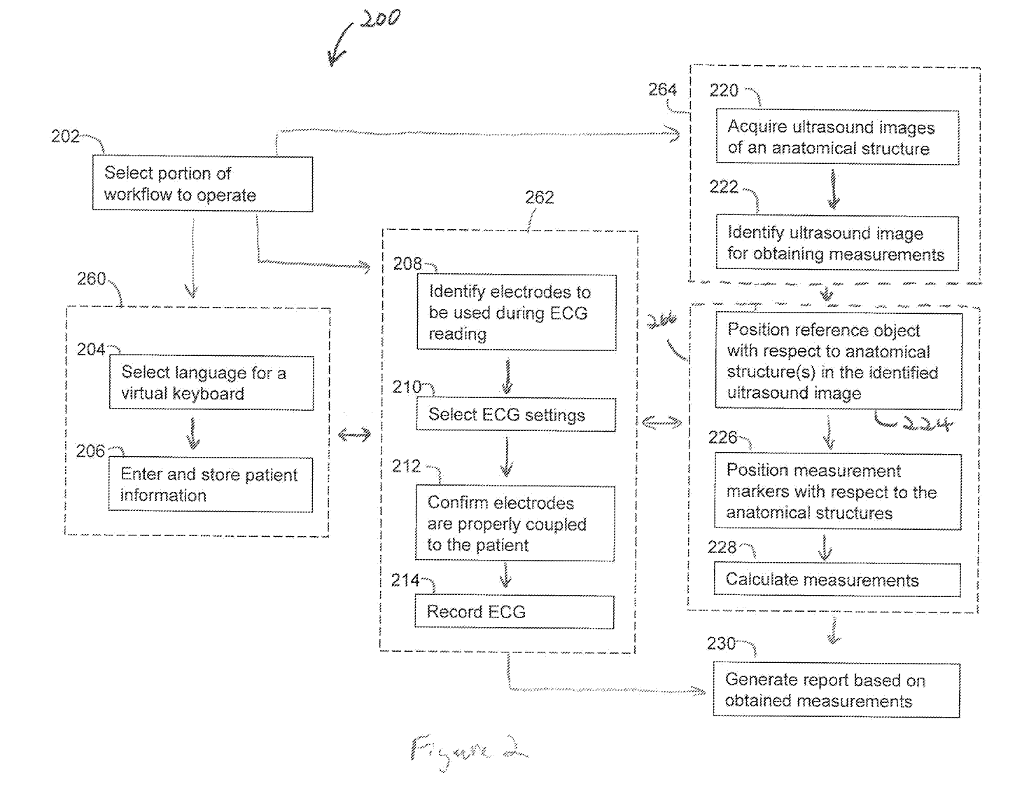

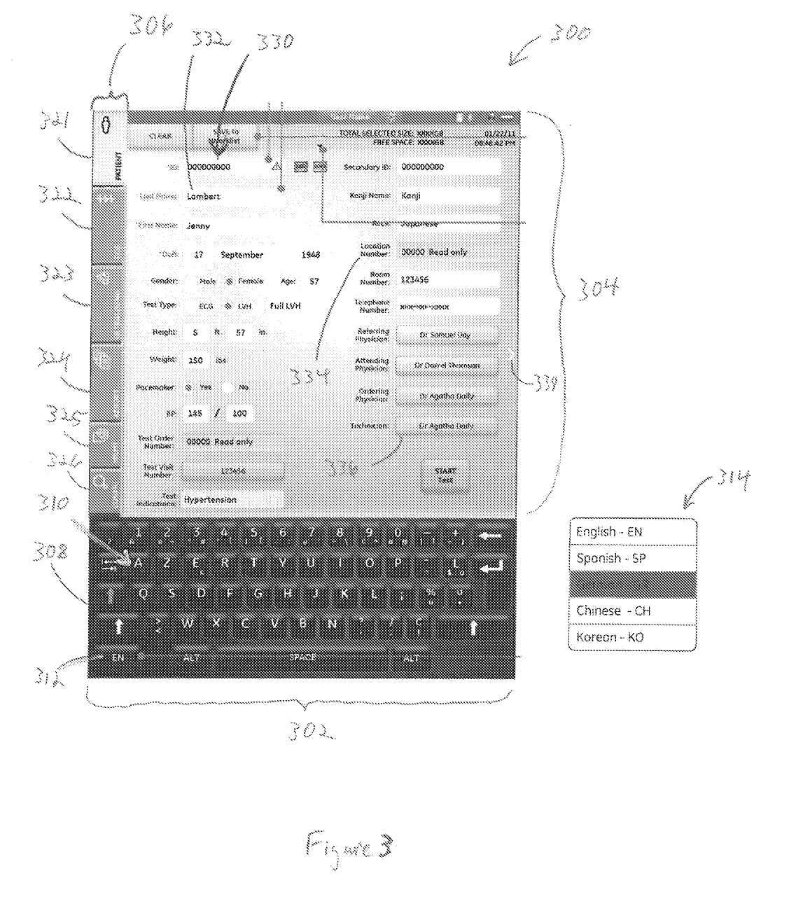

FIG. 2 is a flowchart illustrating a workflow or method 200 in accordance with one embodiment that may be referred to throughout the description of FIGS. 3-11. Although the workflow 200 shows numerous operations or steps that an operator may perform using a diagnostic system, embodiments described herein are not limited to performing each and every operation described herein and/or performing operations in the order shown in FIG. 2. For example, methods may only include operations 222, 224, and 226 (described in greater detail below). Embodiments may also include operations that are not shown in FIG. 2, but are described elsewhere herein. Embodiments described herein are not limited to the order shown in FIG. 2 unless explicitly described otherwise. For example, in alternative embodiments, stage 264 may occur before or occur at least partially simultaneously with stage 262.

FIGS. 3-11 shows various display screens or windows that may be displayed to an operator by one or more embodiments. For example, FIG. 3 shows a demographic screen 300 having a display area that includes a user-input section 302, a patient information section 304, and a workflow-selection section 306. In the illustrated embodiment, the user-input section 302 includes a virtual keyboard 308 that includes a plurality of keys 310 arranged in the AZERTY layout. However, other types of keyboard (or keypads) may be shown with other layouts (e.g., QWERTY, QWERTZ, and others). The virtual keyboard 308 shown in FIG. 3 may operate in a similar manner as a physical keyboard. In an alternative embodiment, the demographic screen 300 does not include a virtual keyboard and, instead, the diagnostic system 100 may include a physical keyboard that is configured to receive and communicate user inputs. In such an alternative embodiment, the patient information section 304 and the workflow-selection section 306 may still be shown in the demographic screen 300.

The workflow 200 may include an administrative stage 260 and a plurality of data-acquisition stages that, in the illustrated embodiment, include an ECG-acquisition stage 262, an ultrasound-acquisition stage 264, and a measurement stage 266. The workflow 200 may include selecting at 202 a portion of the workflow to operate. As shown in FIG. 3, the demographic 300 screen herein may include a plurality of user-selectable elements that include tabs 321-326. The tabs 321-326 are located in the workflow-selection section 306 and are may be activated (e.g., pressed by the operator) to transition between the different stages 260, 262, 264, 266 of the workflow 200. In the illustrated embodiment, the tab 321 is labeled “Patient” and, when activated, may display the demographic screen 300 to the operator in which the operator may view, enter, and/or modify information about a patient for the administrative stage 260. The tab 322 is labeled “ECG” and may be activated when it is desired to obtain an ECG of the patient for the ECG-acquisition stage 262. The tab 323 is labeled “Ultrasound” and may be activated during the diagnostic session when it is desired to obtain ultrasound images of the patient for the ultrasound-acquisition stage 264 and/or to obtain structural measurements for the measurement stage 266. The tab 324 is labeled “Report” and may be activated to generate and/or view a report using the data obtained during at least one of the ECG session or the ultrasound imaging session.

The tabs 325 and 326 are labeled “Mgmt” and “Config,” respectively, and may be used by the operator to perform other functions. For example, the Mgmt (or Management) section may enable the operator to view the progress of transfers, completion status, print and send files, and enable the operator to go back into the workflow to complete a task. The user interface for the Mgmt section may include print logs, transfer logs, system folders, USB folders, and demographic screens. The Config (or Configuration) section may enable the operator to configure other user screens.

The tabs 321-326 enable the operator to move to the different stages 260, 262, 264, 266 of the workflow 200. In some embodiments, the transition may occur at any time. In other words, the operator is not required to follow a particular order of operations. As such, the tabs 321-326 may enable the operator to move between different diagnostic modalities. For example, after acquiring the ultrasound images, the operator may decide to obtain an ECG. The operator may move to the ECG stage 262 of the workflow by pressing the tab 322. As described above, although FIG. 3 shows tabs 321-326, the tabs 321-326 may be other types of user-selectable elements. For example, a single drop-down box may be shown that lists “Patient,” “ECG,” “Ultrasound,” etc. The user may select from the list to move between different portions of the workflow.

The workflow 200 may include selecting at 202 a language setting of the virtual keyboard 308. As shown in FIG. 3, the virtual keyboard 308 includes a language-selection element 312, which is a user-selectable element, shown as one of the keys 310 of the keyboard 308. By activating the language-selection element 312, an operator may move between different keyboards that are based upon different languages. For example, in some embodiments, a menu 314 may appear when the language-selection element 312 is activated. The menu 314 lists a plurality of languages, such as English, Spanish, German, Chinese, and Korean as shown in FIG. 3. The operator may select one of the languages in the menu 314 to change the language setting of the keyboard. For example, in FIG. 3 the letters indicated on the keys 310 are English letters. When the language-selection element 312 is activated, the language setting may change such that the keys 310 have different letters. Moreover, the number and arrangement of the keys 310 may change so that the keyboard 308 is suitable for an operator. For example, the keyboard may change from a QWERTY layout to an AZERTY layout. In other embodiments, the selecting operation at 202 may simply change the keyboard without providing the menu 314. For example, by pressing the language-selection element 312, the English keyboard may change to a German keyboard. Pressing the language-selection element 312 again may change the German keyboard to a Spanish keyboard.

During the administrative stage 260, patient information may be entered and stored at 206. As shown, the demographic screen 300 includes a plurality of fields 330 that are configured to receive data from the operator. Information may be entered into the fields 330 in various manners, such as by selecting the field and typing or by selecting information from a drop-down list. The fields 330 may include patient fields 332 (e.g., last name of patient, first name, date of birth, gender, race, age, height, weight, blood pressure, whether the patient has a pacemaker, and the like), administrator fields 334 (e.g., identification number, secondary identification number, location of diagnostic session, and the like), and test fields 336 (e.g., type of test being performed, referring physician, attending physician, ordering physician, technician, and the like). In some embodiments, the operator may activate a search for patient information by, for example, entering a patient's name and allowing the diagnostic system to search and retrieve the remaining information.

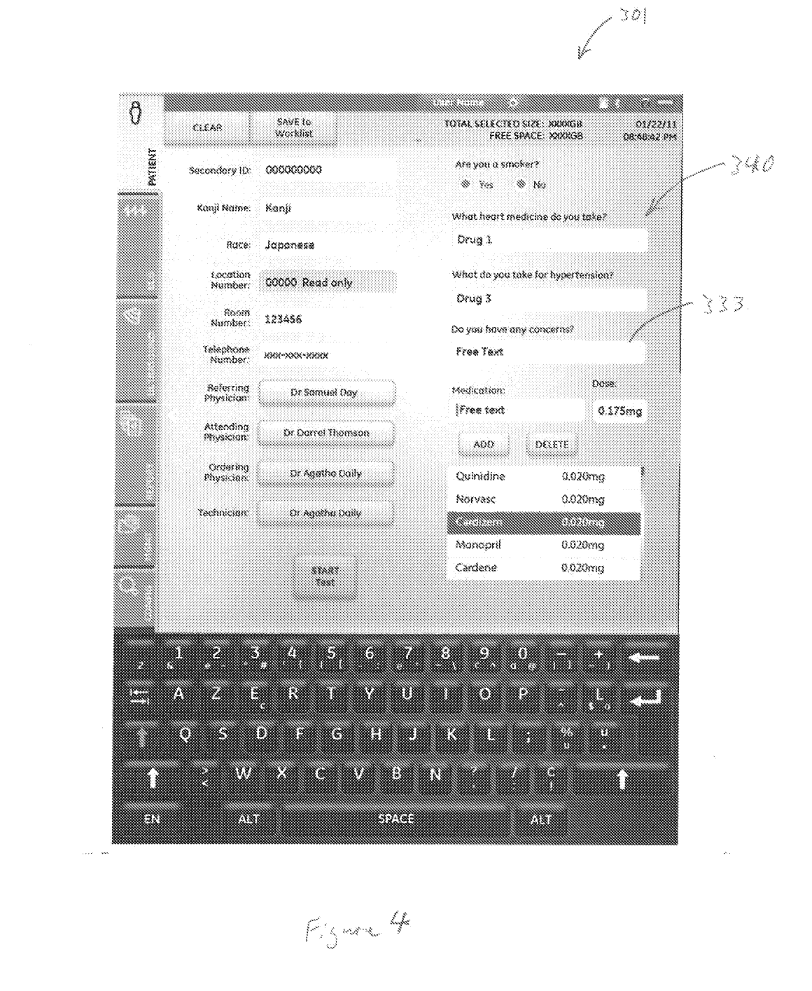

In some embodiments, the demographic screen 300 enables the operator to slide the demographic screen 300 so that the display area changes. For example, the operator may activate a slide element 338 (indicated as an arrowhead) that shifts a view of the demographic screen 300. More specifically, the fields 330 as shown in FIG. 3 may slide to the left so that a new demographic screen 301 shown in FIG. 4 is effectively shown. The demographic screen 301 includes many of the same features of the demographic screen 300. However, the demographic screen 301 includes fields 340 that are not displayed with the demographic screen 300. The fields 340 may be similar to the fields 330 in FIG. 3. Additional patient fields 333 may also be shown by the demographic screen 301. In other embodiments, the demographic screen 300 may transition to the demographic screen 301 without appearing to slide to the next screen.

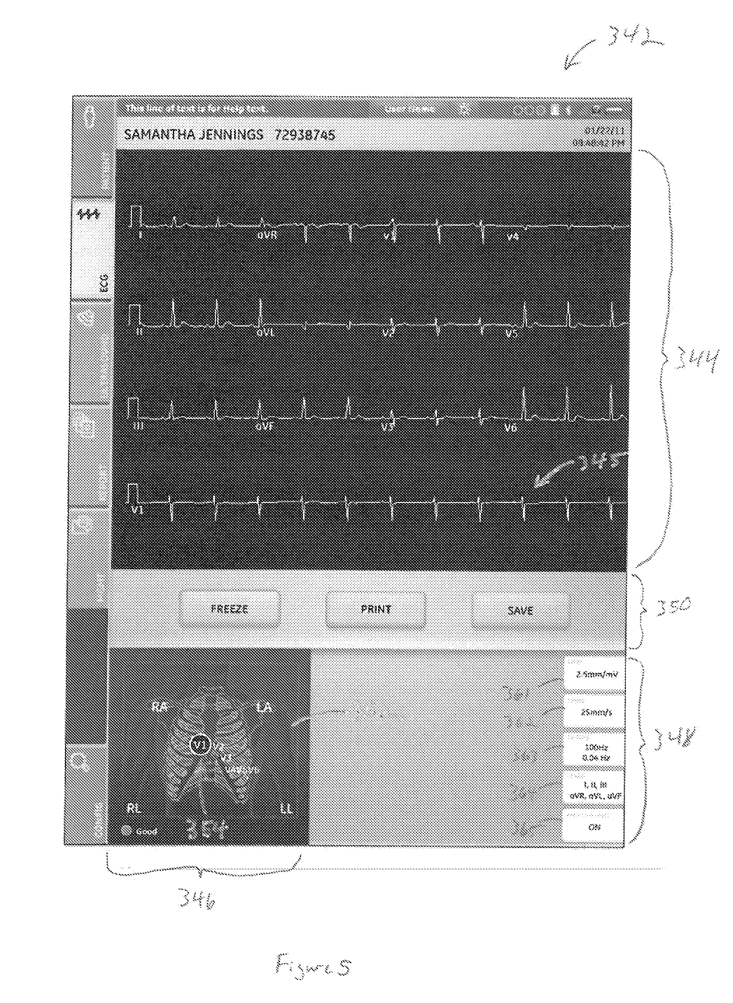

FIG. 5 shows an ECG-acquisition screen 342 that may be presented to the operator during the ECG-acquisition stage 262 of the workflow 200. The ECG-acquisition screen 342 includes a waveform area 344, a lead-advisor portion 346, a data menu 348, and operator controls 350. The lead-advisor portion 346 shows a graphical representation or reference 352 of the patient's body and lead-markers 354 that identify where the electrodes should be located on the patient's body during the ECG. The graphical representation 352 includes a standard torso of a human body. The lead-markers 354 are configured to assist the operator in locating where the electrodes should be positioned on the body of the patient.

As shown in FIG. 5, the lead-marker 354 for “V1” is highlighted on the ECG-acquisition screen 342. In some embodiments, the ECG-acquisition screen 342 may indicate whether an electrode is properly coupled to the patient body or improperly coupled to the patient body (or disconnected). For example, a confirmation signal may be transmitted through the electrodes. The confirmation signal may then be filtered from an ECG signal received by the electrodes. If the confirmation signal is present (and filtered), then the corresponding electrode is determined to be properly coupled to the patient body. If the confirmation signal is not present, then the corresponding electrode is determined to be improperly coupled or disconnected. The lead-markers 354 in the illustrated human body of the graphical representation 352 may indicate whether the electrodes are properly coupled to the patient or improperly coupled to the patient (or disconnected).

In the illustrated embodiment, the waveform area 344 illustrates a 12-lead layout that includes waveforms associated with limb leads I-III; waveforms associated with augmented limb leads aVR, aVL, and aVF; and waveforms associated with chest leads V1-V6. The waveforms show electrical activity of the heart during a predetermined time period from the designated lead. The independent axis (or x-axis) indicates time and the dependent axis (or y-axis) indicates voltage (e.g., in mV). A rhythm strip 345 is shown in the bottom row of the waveform area 344. Each of the limb leads I-III, the augmented leads aVR, aVL, and aVF, and the chest leads V1-V6 receive electrical signals that are transmitted to the diagnostic system 100 and analyzed by the ECG device 106. The waveform generator 166 is configured to process the signals and provide waveforms for each of the electrodes in the waveform area 344 of the ECG-acquisition screen 342. As shown in the ECG-acquisition screen 342, each of the waveforms is located in a portion of the waveform area 344. In some embodiments, the location of the waveforms may be moved by the operator.

The data menu 348 includes user-selectable elements 361-365 that may be activated by the operator to modify the type of data received and/or to modify how the data is displayed. For example, the operator may change the gain (e.g., 2.5 mm/mV, 5 mm/mV, 10 mm/mV, 20 mm/mV, etc.) of the recordings by activating the user-selectable element 361 or speed at which the recordings are transcribed by activating the user-selectable element 362. The operator may also change the filters that are applied to the recordings by activating the user-selectable element 363.

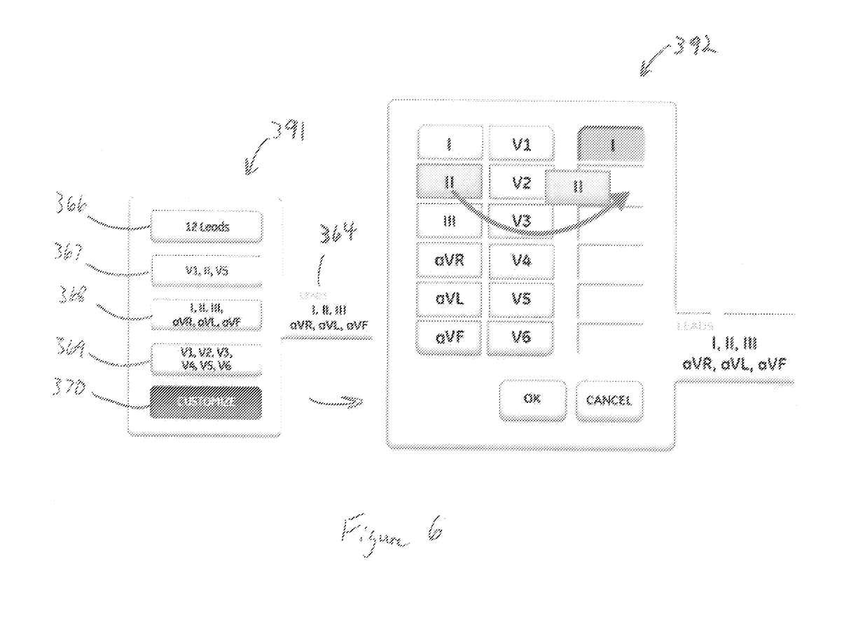

Also shown in FIG. 5, the user-selectable element 364 may be activated to change or modify electrode settings or layouts. For example, FIG. 6 shows GUI elements 391, 392 that may be displayed to the operator when the user-selectable element 364 is activated. For example, when the user-selectable element 364 is activated, the operator may be presented with the GUI element 391 that includes user-selectable elements 366-370 for modifying the layout options of the leads. The layout options include (a) 12 leads at 366; (b) 3 leads at 376 (the 3 leads are selected as V1, II, V5 in FIG. 6); (c) 6 leads at 368 (the 6 leads are selected as I, II, III, aVR, aVL, aVF in FIG. 6); and (d) a user-selected layout option at 369 for one or more leads (the leads are selected as V1-V6 in FIG. 6). User-selectable element 370 is a customization element that enables the operator to customize the layout options (a)-(d) described above. If the operator selects the user-selectable element 370 for a customized layout option, then GUI element 392 may be presented to the operator. As shown, the GUI element 392 enables the operator to select the electrodes that may be used during an ECG recording. For example, a user-selectable element of a desired electrode may be pressed by the operator and moved to an available space to create the desired layout (e.g., a button indicative of the desired electrode may be dragged and dropped into position in the GUI element 392). Once the desired layout has been created, the operator may activate the “OK” button.

Returning to FIG. 5, the operator controls 350 include user-selectable elements (e.g., buttons) that are labeled “FREEZE,” “PRINT,” and “SAVE.” When the ECG device 106 is receiving electrical signals from the patient, the operator may pause recording by activating the FREEZE button. The operator may review the waveforms and decide whether the recordings are satisfactory for analysis. In some embodiments, the diagnostic system 100 may automatically analyze the ECG recording and determine whether the ECG recording is satisfactory. The recording may be satisfactory when the waveforms appear to represent valid electrical readings of a heart. The operator may then activate the PRINT button to print the recording and/or the SAVE button to save the recording to, e.g., a database or other storage unit.

Accordingly, when the operator selects at 202 the ECG tab 322 to enter the ECG-acquisition stage 262, the operator may then customize the ECG reading to be recorded. For example, the operator may identify at 208 the electrodes to be used during the ECG reading and/or select at 210 display options that modify the manner in which the reading is displayed. Before or after the identifying and selecting operations at 208, 210, the operator may couple the electrodes to the patient's body. The diagnostic system 100 may confirm at 212 that the electrodes are properly coupled to the patient body by analyzing electrical signals obtained from the patient. If the electrodes are not receiving signals properly, the operator may re-apply the electrode to the patient body or replace the electrode. At 214, the electrical signals of the patient's heart may be recorded. For example, after viewing the signal recordings for a predetermined period of time (e.g., 10-20 seconds), the operator may activate the FREEZE button to stop the recording. In other embodiments, the system may automatically stop the recording when the system determines that the signal acquired for the predetermined period of time is of a good quality. The readings may then be saved to a storage unit.

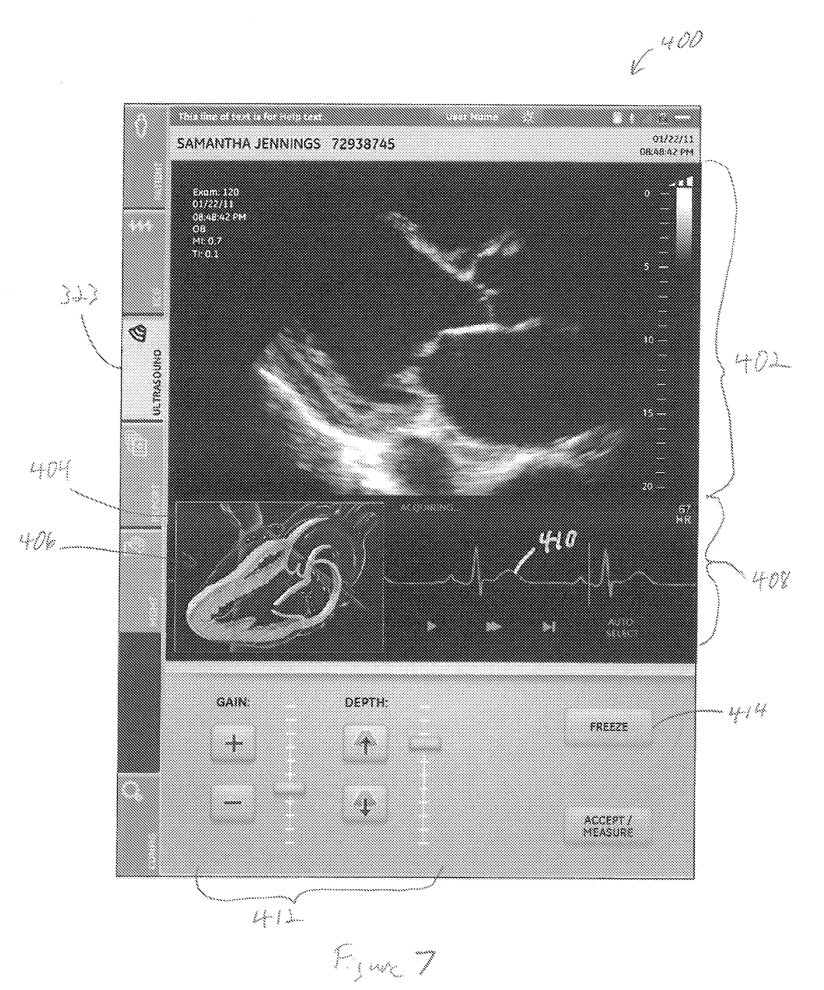

FIG. 7 shows an ultrasound-acquisition screen 400 that may be displayed to the operator when the ULTRASOUND tab 323 is activated and during the ultrasound-acquisition stage 264. The ultrasound-acquisition screen 400 includes an image portion 402 where an ultrasound image is displayed; a reference advisor 404 where a reference illustration 406 of an anatomical structure (e.g., heart) is displayed; a waveform portion 408 where a signal waveform 410 is displayed; and operator controls 412. In an exemplary embodiment, the ultrasound images are B-mode images. As shown, the operator controls 412 enable the operator to change different settings and/or parameters, such as the gain of the ultrasound images and the depth of the ultrasound images shown in FIG. 7. The reference illustration 406 shows a desired orientation of the heart for the ultrasound acquisition. In particular embodiments, the desired orientation of the heart allows the operator to obtain a parasternal long-axis view of the left ventricle. The signal waveform 410 may be obtained by a single ECG electrode that is coupled to the body of the patient. In alternative embodiments, the signal waveform may be obtained by multiple electrodes.

The workflow 200 includes acquiring at 220 ultrasound images of the anatomical structure. For example, during acquisition of the ultrasound images, the operator may activate a user-selectable element 414, which is indicated as a FREEZE button, to capture one or more ultrasound images. In the illustrated embodiment, activation of the user-selectable element 414 may stop image recording and automatically save a predetermine number of ultrasound images prior to activation of the user-selectable element 414. For example, the previous six or ten seconds of ultrasound images may be saved.

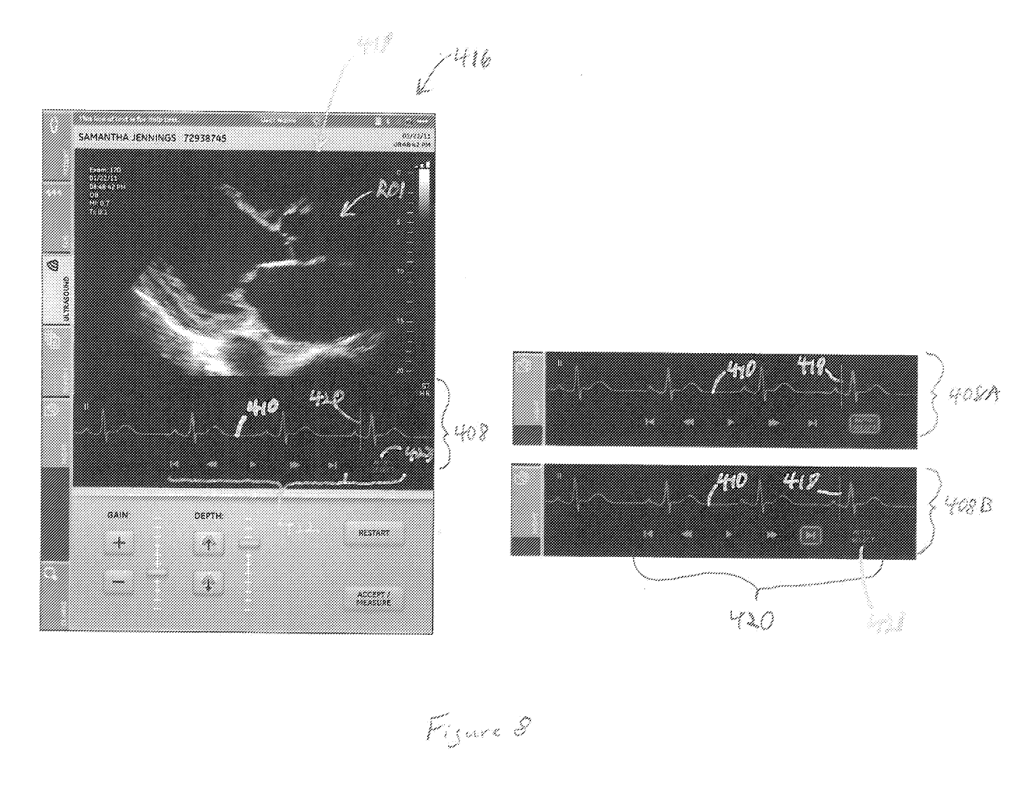

FIG. 8 shows another ultrasound-acquisition screen 416 that may be displayed to the operator. The ultrasound acquisition screen 416 may be similar to the ultrasound-acquisition screen 400, but the reference advisor 404 (FIG. 7) has been removed and the waveform portion 408 may be expanded. The ultrasound-acquisition screen 416 may be displayed when the user-selectable element 414 (FIG. 7) is activated by the operator. In FIG. 8, the signal waveform 410 shows the electrical activity of the patient's heart for four heartbeats.

The signal waveform 410 may be synchronized with the ultrasound images. For example, FIG. 8 shows a single ultrasound image 418 of a region of interest (ROI). In some embodiments, the ROI includes at least a portion of the heart. In particular embodiments, the ultrasound image 418 includes a parasternal long-axis view of the left ventricle (LV). The single ultrasound image 418 directly corresponds to a designated time during ultrasound acquisition. The designated time and, consequently, the single ultrasound image 418 directly corresponds to an electrical measurement along the signal waveform 410. In FIG. 8, a time indicator 420 is located at the designated time on the signal waveform 410. The time indicator 420 is illustrated as a vertical line and may have a color that differs from a color of the signal waveform. However, other GUI elements may be used to indicate time.

The workflow 200 may also include identifying at 222 an ultrasound image for obtaining measurements. For example, when the ROI includes a heart, the identified ultrasound image may show the heart at a predetermined moment during the cardiac cycle. To this end, when the user-selectable element 414 is activated, time-selection elements 422 may appear with the signal waveform 410. As shown in FIG. 8, the time-selection elements 422 include user-selectable elements. The time-selection elements 422 include an AUTO-SELECT element 423 that instructs the diagnostic system to automatically identify the desired ultrasound image. For example, the cardiac cycle analyzer 124 may analyze the ultrasound images of the recording and/or the signal waveform 410 to identify a designated ultrasound frame (e.g., the single ultrasound image 418) that is associated with a designated moment of the cardiac cycle. For instance, the cardiac cycle analyzer 124 may analyze the anatomical structures shown in the ultrasound images to identify when the anatomical structures have a predetermined relationship with respect to each other. More specifically, the cardiac cycle analyzer 124 may analyze the movements of heart walls and valves and the change in chamber size to identify different stages in the heart cycle. In particular embodiments, the cardiac cycle analyzer 124 identifies an ultrasound image that corresponds to an end diastole of the cardiac cycle.

After the cardiac cycle analyzer 124 has automatically identified an ultrasound image that is associated with a predetermined moment in the heart cycle, the operator may use the time-selection elements 422 to confirm or verify that the ultrasound image identified by the cardiac cycle analyzer 124 is the desired ultrasound image. For example, the time-selection elements 422 also include virtual buttons that are similar to buttons of a video-cassette recorder (VCR) or DVD player. The time-selection elements 422 may enable the operator to forward, fast-forward, rewind, fast-rewind, and play the combined ultrasound/ECG recording. When the time indicator 420 is moved to a selected time, the ultrasound image shown in the ultrasound-acquisition screen 416 is changed to the ultrasound image that is associated with the selected time. Accordingly, the time-selection elements 422 may permit the operator to scan or move the time indicator 420 along the signal waveform 410 thereby changing the ultrasound image to confirm/identify/select the ultrasound image that is most representative of the predetermined moment in the heart cycle.

By way of example, the imaging device 108 may be capable of imaging at 50 frames/second. In such embodiments, each ultrasound image 418 may correspond to 0.02 seconds. Accordingly, the time indicator 418 may be moved along the x-axis of the signal waveform 410 in incremental steps that correspond to 0.02 seconds. In some cases, the ultrasound images before or after the ultrasound image identified by the cardiac cycle analyzer 124 may be a better representation of the predetermined moment in the cardiac cycle that is desired by the operator. The operator may then select the appropriate ultrasound image by selecting the user-selectable element 422, which is a button that is labeled ACCEPT/MEASURE.

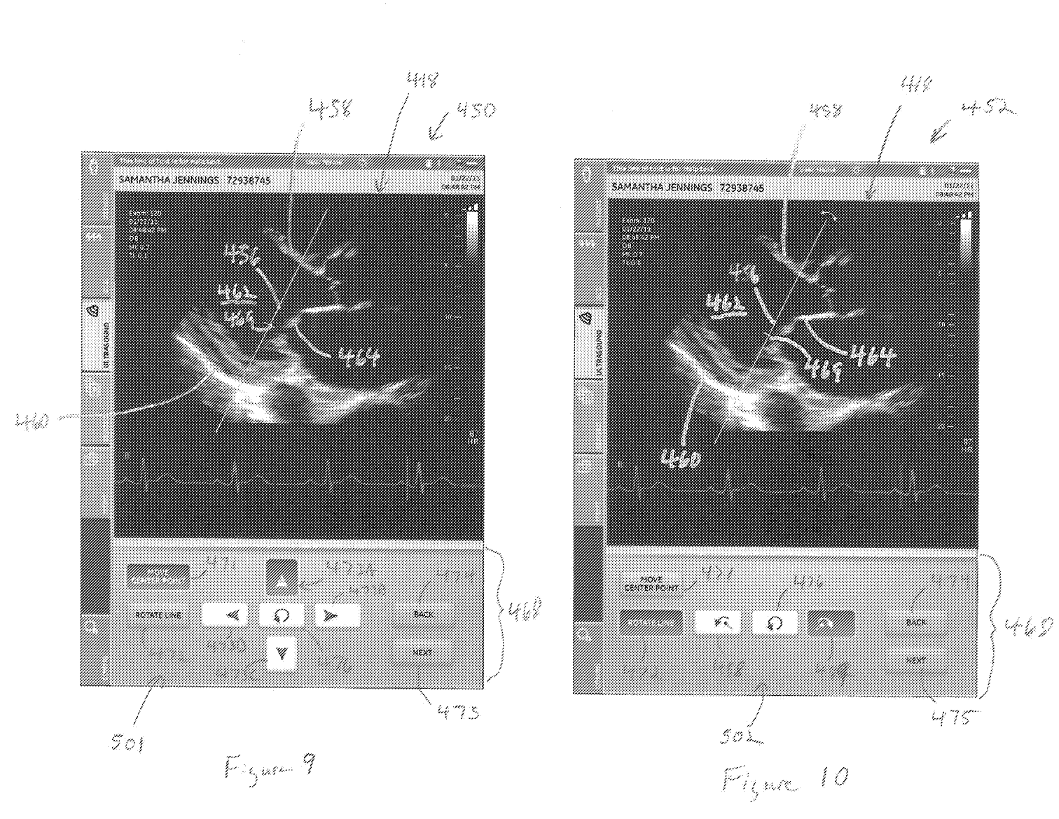

FIGS. 9-11 illustrate respective measurement screens 450, 452, and 454 that may be shown to the operator during the measurement stage 266. The workflow 200 may include positioning at 224 a reference object 456 on the ultrasound image 418. As will be described in greater detail below, the reference object 456 may be used to obtain measurements (e.g., values of different dimensions) of anatomical structures 458, 460 shown in the ultrasound image 418. In the illustrated embodiment, the reference object 456 is a projection line and, as such, may be referred to as the projection line 456 hereinafter. The illustrated anatomical structures 458, 460 include an inter-ventricular septal wall and a posterior wall, respectively, of the patient's heart and may also be referred to as the septal wall 458 and the posterior wall 460 hereinafter. Also shown in FIGS. 9-11, a chamber 462 is located between the septal and posterior walls 458, 460. A third anatomical structure 464, the atrioventricular (mitral) valve (also referred to as a bicuspid valve), is also shown in FIGS. 9-11. The mitral valve 464 is located between the left ventricle and the left atrium.

The positioning operation 224 may include multiple stages or sub-operations for positioning the reference object 456. For example, with respect to FIG. 9, a first stage may include automatically positioning the reference object 456 with respect to the anatomical structures 458, 460. In the exemplary embodiment, the projection line 456 has a center point 469 that the projection line 456 is configured to be rotated about. The measurement module 125 may analyze the ultrasound image 418 to identify one or more features of the heart shown in the ultrasound image 418, such as at least one of the septal wall 458, the posterior wall 460, the chamber 462, or the mitral valve 464. The measurement module 125 may automatically position the center point 469 within the chamber 462 between the septal and posterior walls 458, 460 and proximate to the mitral valve 464. The measurement module 125 may also orient the projection line 456 such that the projection line 456 intersects the septal and posterior walls 458, 460 in a substantially perpendicular manner.

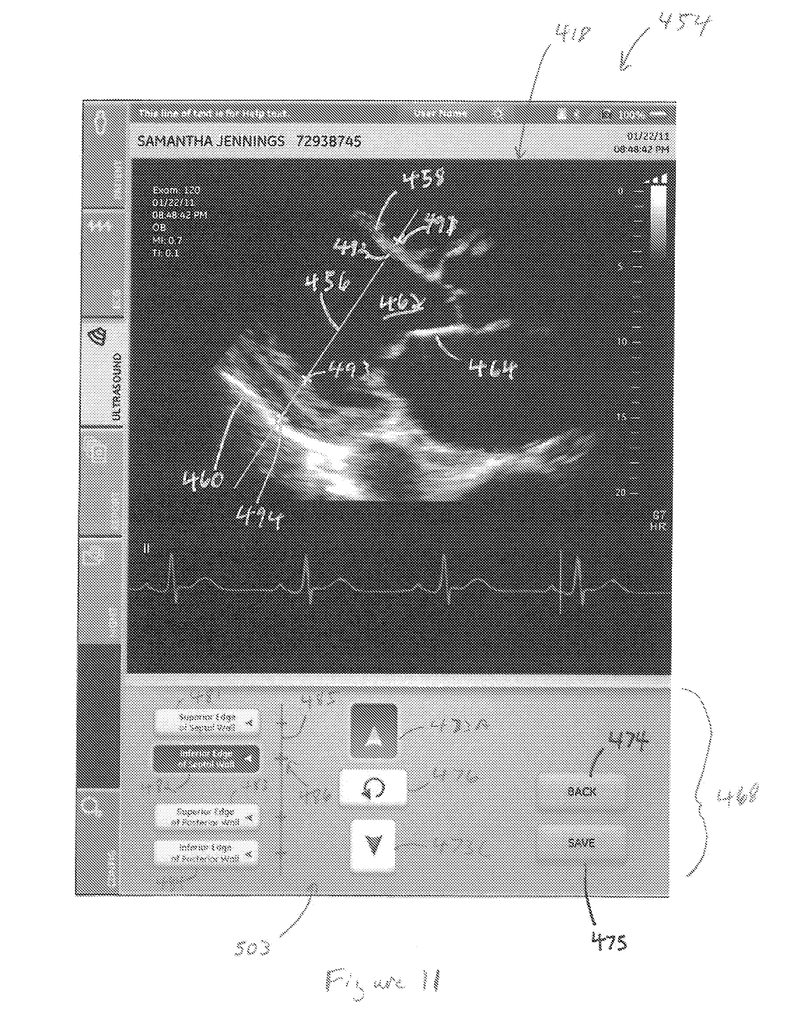

In FIG. 9, the user-selectable element 471 labeled MOVE CENTER POINT is indicated as being activated. The positioning operation 224 may also include receiving operator inputs to modify the position of the projection line 456. The measurement screen 450 includes a control portion 468 that includes user-selectable elements 471-476, which include center point locators 473A-473D. The center point locators 473A-473D are shown as four arrow keys (up, down, left, right) in FIG. 9 that, when activated, enable the operator to move the center point 469 of the projection line 456. When the center point 469 is moved, the remainder of the projection line 456 may follow the center point 469. The user-selectable element 476 is an “undo” feature that enables the operator to return to a previous setting, such as a previous position of the projection line 456 before the projection line was moved. Once the operator has confirmed that the position of the center point 469 is sufficient, the operator may activate the user-selectable element 475 labeled NEXT to transition to the measurement screen 452 shown in FIG. 10.

With respect to FIG. 10, the positioning operation 224 may also include receiving operator inputs to modify an orientation (or rotation) of the projection line 456. For example, the measurement screen 452 includes the control portion 468 that has user-selectable elements 488 and 489 in addition to the user-selectable elements 472, 474, 475, and 476. The user-selectable elements 488, 489 may be referred to as rotating elements that enable the operator to rotate the projection line 456 about the center point 469. The user-selectable element 488 allows the operator to rotate the projection line 456 in a counter-clockwise direction, and the user-selectable element 489 allows the operator to rotate the projection line 456 in a clockwise direction. Once the operator has confirmed that the rotation of the projection line 456 is sufficient, the operator may activate the user-selectable element 475 to transition to the measurement screen 454.

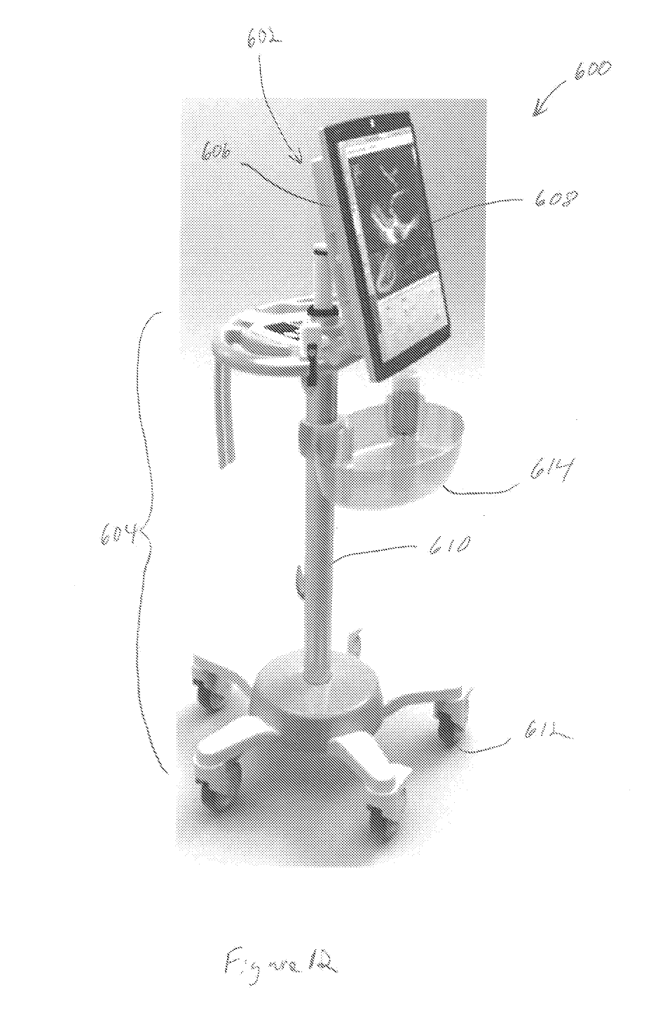

As described above, one or more embodiments described herein are configured to obtain one or more measurements (e.g., dimensions of anatomical structures, ECG recordings) from a patient. The obtained measurements may then be analyzed by the diagnostic system and/or a healthcare provider to diagnose a medical condition of the patient. To this end, the workflow 200 may also include positioning at 226 measurement markers 491-494 for measuring anatomical structures in the ultrasound image. In FIG. 11, the measurement screen 454 includes the control portion 468. The control portion 468 includes the user-selectable elements 473A, 473C, and 474-476. The control portion 468 also includes user-selectable elements 481-484. Similar to the object-positioning operation 224, the marker-positioning operation 226 may include multiple stages or sub-operations for locating the measurement markers 491-494.

For example, the marker-positioning operation 226 may include automatically locating the measurement markers 491-494 with respect to the anatomical structures 458, 460. In the illustrated embodiment, the measurement marker 491 is configured to be positioned on the superior edge of the septal wall 458; the measurement marker 492 is configured to be positioned on the inferior edge of the septal wall 458; the measurement marker 493 is configured to be positioned on the superior edge of the posterior wall 460; and the measurement marker 494 is configured to be positioned on the inferior edge of the posterior wall 460.

To automatically locate the measurement markers 491-494 on the ultrasound image 418, the measurement module 125 may analyze the ultrasound image 418 and, more particularly, the anatomical structures 458, 460 to determine where the superior and inferior edges of the septal wall 458 are located and where the superior and inferior edges of the posterior wall 460 are located. The measurement module 125 may use, for example, edge-detection algorithms and, optionally, stored data that may inform the measurement module 125 as to where the edges are typically located for a heart. For example, the measurement module 125 may analyze the pixel intensities of the pixels in the ultrasound image proximate to the areas where the projection line 456 intersects the septal and posterior walls 458, 460. After determining where the edges are located, the measurement module 125 may position the markers 491-494 at the respective locations.

However, in some embodiments, the diagnostic system 100 enables the operator to move the measurement markers 491-494 from the automatically determined locations. Accordingly, the marker-positioning operation 226 may include receiving operator inputs to move at least one of the measurement markers 491-494. In some embodiments, the markers 491-494 may be moved individually by the operator. For example, the user-selectable elements 481-484 (also called marker elements) are labeled, respectively, “Superior Edge of Septal Wall,” “Inferior Edge of Septal Wall,” “Superior Edge of Posterior Wall,” and “Inferior Edge of Posterior Wall.” If the operator desires to move any one of the measurement markers 491-494, the operator may activate the appropriate marker element and utilize the user-selectable elements 473A and 473C to move the corresponding marker along the projection line 456. For example, the user-selectable element 482 is indicated as being activated in FIG. 11. When the user-selectable element 482 is activated, the operator is enabled to move the measurement marker 492 along the projection line 456. In the illustrated embodiment, the measurement markers 491-494 are only moved along the projection line 456 (e.g., up or down the projection line 456) to the desired location. In other embodiments, the markers 491-494 are not limited to locations along the projection line 456.

To facilitate the operator in identifying the measurement marker that is being moved (also referred to as the “movable marker”), an appearance of the movable marker may be altered to indicate to the operator that the movable marker is capable of being moved by the user-selectable elements 473A and 473C. By way of example, in the illustrated embodiment, the user-selectable element 482 is activated. The measurement marker 492 is indicated in a different color as compared to when the user-selectable element 482 is not activated. For example, the measurement marker 492 may be pink or yellow, whereas the markers 491, 493, and 494 may be gray. The measurement marker 492 may also be gray when the user-selectable element 482 is not activated.

Moreover, the control portion 468 may include a representative line 485 having representative markers 486 located therealong. Each of the representative markers 486 is associated with a corresponding one of the user-selectable elements 481-484 and one of the measurement markers 491-494. The representative markers 486 may have a similar appearance (e.g., size, shape, and color) to the corresponding measurement markers 491-494. For example, when the user-selectable element 482 is activated as shown in FIG. 11, the representative marker 486 associated with the user-selectable element 482 and the measurement marker 492 may have a similar appearance. In the illustrated embodiment, the representative marker 486 associated with the user-selectable element 482 and the measurement marker 492 have the same size, shape, and color and are distinguishable from the other markers.

In some embodiments, the measurement markers 491-494 are configured to indicate a localized point within the ultrasound image 418. As shown, the markers 491-494 are illustrated as cross-hairs. However, alternative markers may have alternative structures (e.g., size, shape, configurations) as well as other colors. For example, the markers may be dots, circles, triangles, arrows, and the like that indicate to the operator a particular location. In other embodiments, the markers 491-494 do not indicate a localized point but a larger area within the ultrasound image. For example, the markers 491-494 may be circles with a large diameter or circumference.

As shown by comparing FIGS. 9-11, the control portion 468 may change as the operator moves between the measurement screens 450, 452, and 454. The measurement screens 450, 452, 454 may have different arrangements of user-selectable elements to guide the operator during the measurement stage 266. In some cases, at least one of the user-selectable elements remains unchanged as the operator moves from one measurement screen to the next. For example, the measurement screen 450 has a first arrangement 501 that includes the user-selectable elements 471, 472, 473A-473D, and 474-476. The measurement screen 452 has a second arrangement 502 that includes the user-selectable elements 471, 472, 474, 475, and 476, which are shared by the first arrangement. However, the second arrangement 502 does not include some of the user-selectable elements in the first arrangement (e.g., the user-selectable elements 473A-473D). The second arrangement 502 also includes user-selectable elements 488 and 489. The third arrangement 503 of the control portion 468 is shown in FIG. 11 and includes the user-selectable elements 473A, 473C, 476, and 474. The third arrangement 503 does not include at least some of the user-selectable elements in the first and second arrangements. However, the third arrangement includes user-selectable elements 481-484.

Accordingly, when the operator moves from one measurement screen to the next, the arrangement of user-selectable elements may change. The change in the arrangement may facilitate guiding the operator by indicating to the operator what functionalities are available in the present measurement screen. By way of example, the first arrangement 501 in the measurement screen 450 indicates to the operator that the center point 469 may be moved in different x-y directions along the ultrasound image 418. The second arrangement 502 in the measurement screen 452 indicates to the operator that the projection line 456 may be rotated about the center point 469. The third arrangement 503 indicates to the operator that the different markers 491-494 on the projection line 456 may be individually moved by the operator by activating one of the user-selectable elements 481-484. In such instances, the diagnostic system 100 provides a user-friendly interface that guides the operator along the various steps for determining different measurements.

The structural measurements may be calculated at 228. For example, the measurement module 125 may measure a distance between the markers 491 and 492. The measured distance may be representative of a septal wall thickness. The measurement module 125 may also measure a distance between the markers 493 and 494. The measured distance may be representative of a posterior wall thickness. In some embodiments, the measurement module 125 may also measure a distance between the markers 492 and 493, which may represent a chamber diameter. In some embodiments, the measurement module 125 may calculate other measurements based on the obtain measurements. For example, the measurement module 125 may calculate a LV mass.

After the workflow data is obtained (e.g., ECG and dimensions of anatomical structures), the workflow may also include generating at 230 a report. The report is based upon the obtained measurements and may simply provide those measurements. However, in other embodiments, the report may include a recommended diagnosis regarding a medical condition of interest. The report generator 126 may analyze various data, including the measurements, and determine whether the patient has a medical condition, such as LVH. For example, the measurements may include at least one of an LV mass, septal wall thickness, or posterior wall thickness. The ECG may include electrical abnormalities (e.g., in the PQRST waveform) that are indicative of the medical condition of interest. The report generator 126 may analyze at least one of the LV mass, the septal wall thickness, the posterior wall thickness, and/or the ECG to diagnose the medical condition of interest for the patient. For example if at least one of the LV mass, the septal wall thickness, or the posterior wall thicknesses exceed a designated value and/or if the ECG includes one more abnormalities, the report generator 126 may generate a report that diagnoses the patient with the medical condition.

It should be noted that FIGS. 9-11 merely illustrate one example of embodiments described herein in which a heart is imaged and dimensions of the different structures in the heart are determined. However, other anatomical systems, organs, or structures of a patient body may be analyzed to determine measurements thereof. Moreover, in alternative embodiments, the reference object may have other geometric shapes that an operator may use as a reference or standard for obtaining measurements of anatomical structures.

FIG. 12 is a perspective view of a portable diagnostic system 600 formed in accordance with one embodiment. The diagnostic system 600 may be similar to the diagnostic system 100 (FIG. 1) and include similar features. In the illustrated embodiment, the diagnostic system 600 includes a workstation or console 602 and a movable carrier 604 that supports the workstation 602. The workstation 602 is configured to be communicatively coupled to an ultrasound probe (not shown) and/or one or more electrodes (not shown) configured to obtain electrical data from a patient. The workstation 602 includes a system body or housing 606 that holds a computing system (not shown) and base units (not shown) of an ECG device and an ultrasound imaging device. The computing system and base units may be similar to the computing system 102 and the base units 112, 116 shown in FIG. 1. The workstation 602 also includes a display 608 that may be part of a user interface of the diagnostic system 600. The display 608 is a touch-sensitive display and may operate in a similar manner as the display 110 described above. As shown, the diagnostic system 600 enables an individual (e.g., the operator) to move the workstation 602 using the carrier 604. The carrier 604 may include a post or stand 610 and a plurality of wheels 612 for moving the workstation 602. The carrier 604 may also include a basket 614 for holding various components, such as the probe and the leads.

A technical effect of the various embodiments of the systems and methods described herein include user-friendly interfaces for obtaining structural measurements of an anatomical structure(s) in a patient body. The user interface may also direct or guide the operator throughout a workflow to obtain the desired data (e.g., electrical and ultrasound data). Another technical effect may be the generation of a report that assists a qualified individual (e.g., doctor) in diagnosing a cardiac medical condition (e.g., LVH) of a patient. Other technical effects may be provided by the embodiments described herein.

As described above, the various components and modules described herein may be implemented as part of one or more computers or processors. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor. The instructions may be stored on a tangible and/or non-transitory computer readable storage medium coupled to one or more servers.

As used herein, the term “computer” or “computing system” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “computing system.”

The set of instructions may include various commands that instruct the computer or processor as a processing machine to perform specific operations such as the methods and processes described herein. The set of instructions may be in the form of a software program. The software may be in various forms such as system software or application software. Further, the software may be in the form of a collection of separate programs, a program module within a larger program or a portion of a program module. The software also may include modular programming in the form of object-oriented programming. The processing of input data by the processing machine may be in response to user commands, or in response to results of previous processing, or in response to a request made by another processing machine. The program is complied to run on both 32-bit and 64-bit operating systems. A 32-bit operating system like Windows XP™ can only use up to 3 GB bytes of memory, while a 64-bit operating system like Window's Vista™ can use as many as 16 exabytes (16 billion GB).

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

In one embodiment, a medical diagnostic system is provided that includes an electrocardiograph (ECG) device having at least one electrode that is configured to obtain electrical data for a heart of a patient. The diagnostic system also includes an ultrasound imaging device having an ultrasound probe that is configured to obtain ultrasound data of the heart of the patient. The diagnostic system also includes a user interface having a display. The user interface is configured to receive operator inputs from an operator of the diagnostic system, wherein the user interface is configured to show on the display a plurality of different screens to the operator during a workflow. The screens include user-selectable elements that are configured to be activated by the operator during the workflow. The user interface is configured to display the different screens to guide the operator through the workflow to obtain the electrical data and the ultrasound data. The user interface also configured to guide the operator to obtain structural measurements of the heart based on the ultrasound data.

In another aspect, the display is a touch-sensitive display having a display area. The touch-sensitive display is configured to detect and identify a location of a touch from the operator.

In another aspect, the plurality of different screens include first and second measurement screens. The first measurement screen is configured to display an ultrasound image and a projection line that is located relative to the ultrasound image. The second measurement screen is configured to display markers that are arranged on the projection line. The first measurement screen may include user-selectable elements that are configured to be activated by the operator to move the projection line. The second measurement screen may include user-selectable elements that are configured to be activated by the operator to move the markers along the projection line.

In another aspect, the plurality of different screens include an ultrasound-acquisition screen. The ultrasound-acquisition screen includes user-selectable elements that enable the operator to view a series of ultrasound images to identify a cardiac-cycle image of the heart, wherein the cardiac-cycle image includes the heart at a predetermined cardiac-cycle event.

In another aspect, the workflow includes generating a report that diagnoses a medical condition of the patient. The report may be based on the electrical data and the structural measurements of the heart.

In one embodiment, a medical diagnostic system is provided that includes an ultrasound imaging device having an ultrasound probe that is configured to obtain ultrasound data of a heart of a patient. The diagnostic system also includes a cardiac cycle analyzer that is configured to analyze the ultrasound data to automatically identify a cardiac-cycle image from a set of ultrasound images based on the ultrasound data. The cardiac-cycle image includes the heart at a predetermined cardiac-cycle event. The diagnostic system also includes a measurement module that is configured to analyze the cardiac-cycle image and automatically position a reference object relative to at least one anatomical structure of the heart in the cardiac-cycle image. The diagnostic system also includes a user interface having a display configured to display the reference object and the cardiac-cycle image. The user interface is configured to receive operator inputs to at least one of (a) designate, from the set of ultrasound images, a different ultrasound image as the cardiac-cycle image or (b) re-position the reference object relative to the at least one anatomical structure.

In one aspect, the measurement module is configured to determine at least one measurement of the heart based on the reference object.

In another aspect, the diagnostic system also includes an electrocardiograph (ECG) device configured to obtain an ECG from the patient and a diagnosis module, the diagnosis module configured to analyze the ECG and the at least one measurement of the heart to determine whether the patient has a medical condition. The medical condition may be left ventricular hypertrophy (LVH).

In another aspect, the user interface is configured to receive user inputs to position first and second measurement markers with respect to the heart in the cardiac-cycle image. The diagnostic system is configured to determine a dimension of the heart that is measured between the first and second measurement markers.

In another aspect, the display is configured to display user-selectable elements that are configured to be activated by the operator to re-position the reference object relative to the at least one anatomical structure.

In another aspect, the display is configured to display first and second screens having first and second arrangements of user-selectable elements, respectively. Each of the first and second screens includes the cardiac-cycle image. The first and second arrangements of the user-selectable elements are different and are configured to guide the operator in re-positioning the reference object relative to the at least one anatomical structure.

In another aspect, the reference object is a projection line that is configured to intersect the heart in the cardiac-cycle image. The projection line may include a center point, and the user interface may be configured to receive operator inputs to at least one of (1) move the center point of the projection line with respect to the heart or (2) rotate the projection line about the center point.

In another aspect, the predetermined cardiac-cycle event is an end diastole of the cardiac cycle. In another aspect, at least one anatomical structure of the heart includes a septal wall and a posterior wall of a left ventricle of the heart. In another aspect, the display is a touch-sensitive display.

In another embodiment, a method of obtaining measurements of a heart of a patient is provided. The method includes automatically identifying a cardiac-cycle image from a set of ultrasound images. The cardiac-cycle image includes the heart at a predetermined cardiac-cycle event. The method also includes displaying the cardiac-cycle image to an operator using a user interface display. The method also includes automatically positioning a reference object relative to at least one anatomical structure of the heart in the cardiac-cycle image. The reference object is positioned to obtain designated measurements of the heart. The method also includes receiving operator inputs from the operator to at least one of (a) designate, from the set of ultrasound images, a different ultrasound image as the cardiac-cycle image or (b) re-position the reference object relative to the at least one anatomical structure. The method also includes determining at least one measurement of the heart using the reference object and the cardiac-cycle image.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A medical diagnostic system comprising:
an electrocardiograph (ECG) device including at least one electrode that is configured to obtain electrical data for a heart of a patient;
an ultrasound imaging device including an ultrasound probe that is configured to obtain ultrasound data of the heart of the patient; and
a user interface comprising a display, the user interface configured to receive operator inputs from an operator of the diagnostic system, wherein the user interface is configured to show on the display a plurality of different screens to the operator during a workflow, the screens including user-selectable elements that are configured to be activated by the operator during the workflow, wherein the user interface is configured to display the different screens to guide the operator through the workflow to obtain the electrical data and the ultrasound data, the user interface also configured to guide the operator to obtain structural measurements of the heart based on the ultrasound data.

2. The diagnostic system of claim 1, wherein the display is a touch-sensitive display having a display area, the touch-sensitive display configured to detect and identify a location of a touch from the operator.

3. The diagnostic system of claim 1, wherein the plurality of different screens include first and second measurement screens, the first measurement screen configured to display an ultrasound image and a projection line that is located relative to the ultrasound image, the second measurement screen configured to display markers that are arranged on the projection line.

4. The diagnostic system of claim 3, wherein the first measurement screen includes user-selectable elements that are configured to be activated by the operator to move the projection line.

5. The diagnostic system of claim 3, wherein the second measurement screen includes user-selectable elements that are configured to be activated by the operator to move the markers along the projection line.

6. The diagnostic system of claim 1, wherein the plurality of different screens include an ultrasound-acquisition screen, the ultrasound-acquisition screen including user-selectable elements that enable the operator to view a series of ultrasound images to identify a cardiac-cycle image of the heart, wherein the cardiac-cycle image includes the heart at a predetermined cardiac-cycle event.

7. The diagnostic system of claim 1, wherein the workflow includes generating a report that diagnoses a medical condition of the patient, the report being based on the electrical data and the structural measurements of the heart.

8. A medical diagnostic system comprising:
an ultrasound imaging device including an ultrasound probe that is configured to obtain ultrasound data of a heart of a patient;
a cardiac cycle analyzer configured to analyze the ultrasound data to automatically identify a cardiac-cycle image from a set of ultrasound images based on the ultrasound data, wherein the cardiac-cycle image includes the heart at a predetermined cardiac-cycle event;
a measurement module configured to analyze the cardiac-cycle image and automatically position a reference object relative to at least one anatomical structure of the heart in the cardiac-cycle image; and
a user interface comprising a display configured to display the reference object and the cardiac-cycle image, wherein the user interface is configured to receive operator inputs to at least one of (a) designate, from the set of ultrasound images, a different ultrasound image as the cardiac-cycle image or (b) re-position the reference object relative to the at least one anatomical structure.

9. The diagnostic system of claim 8, wherein the measurement module is configured to determine at least one measurement of the heart based on the reference object and wherein the diagnostic system further comprises an electrocardiograph (ECG) device and a report generator, the ECG device configured to obtain an ECG from the patient, the report generator configured to analyze the ECG and the at least one measurement of the heart to determine whether the patient has a medical condition.

10. The diagnostic system of claim 9, wherein the medical condition is left ventricular hypertrophy (LVH).

11. The diagnostic system of claim 8, wherein the user interface is configured to receive user inputs to position first and second measurement markers with respect to the heart in the cardiac-cycle image, wherein the diagnostic system is configured to determine a dimension of the heart that is measured between the first and second measurement markers.

12. The diagnostic system of claim 8, wherein the display is configured to display user-selectable elements that are configured to be activated by the operator to re-position the reference object relative to the at least one anatomical structure.

13. The diagnostic system of claim 8, wherein the display is configured to display first and second screens having first and second arrangements of user-selectable elements, respectively, each of the first and second screens including the cardiac-cycle image, wherein the first and second arrangements of the user-selectable elements are different and are configured to guide the operator in re-positioning the reference object relative to the at least one anatomical structure.

14. The diagnostic system of claim 8, wherein the reference object is a projection line that is configured to intersect the heart in the cardiac-cycle image.

15. The diagnostic system of claim 8, wherein the predetermined cardiac-cycle event is an end diastole of the cardiac cycle.

16. The diagnostic system of claim 8, wherein the display is a touch-sensitive display.

17. A method of obtaining measurements of a heart of a patient, the method comprising:
automatically identifying a cardiac-cycle image from a set of ultrasound images, wherein the cardiac-cycle image includes the heart at a predetermined cardiac-cycle event;
displaying the cardiac-cycle image to an operator using a user interface display;
automatically positioning a reference object relative to at least one anatomical structure of the heart in the cardiac-cycle image, the reference object being positioned to obtain designated measurements of the heart;
receiving operator inputs from the operator to at least one of (a) designate, from the set of ultrasound images, a different ultrasound image as the cardiac-cycle image or (b) re-position the reference object relative to the at least one anatomical structure; and
determining at least one measurement of the heart using the reference object and the cardiac-cycle image.

18. The method of claim 17, further comprising analyzing an electrocardiograph of the heart and the at least one measurement of the heart to determine whether the patient has a medical condition.

19. The method of claim 18, wherein the medical condition is left ventricular hypertrophy (LVH).

20. The method of claim 17, wherein the receiving operator inputs includes receiving operator inputs to position first and second measurement markers with respect to the heart in the cardiac-cycle image.
